(19) United States
(12) Patent Application Publication
(10) Pub. No.: US 2008/0090238 A1
(43) Pub. Date: Apr. 17, 2008

(54) Increased sensitivity of proximity ligation assays

(76) Inventors: Dan-Hui Dorothy Yang, Sunnyvale, CA (US); Brian Jon Peter, Los Altos, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/581,191

(22) Filed: Oct. 12, 2006

(51) Int. Cl. C12Q 1/68; C12P 19/34

(52) U.S. Cl. 435/6; 435/91.2

(57) ABSTRACT

Methods for enhancing the sensitivity of proximity ligation assays are provided herein. The methods make use of size separation methods, control of oligonucleotide size, and control of reaction conditions, to improve the assay sensitivity. Kits for performing the assay are also described.

### BACKGROUND

Sensitive and specific protein detection and analyses have been of great interest in biological and medical science. In contrast to methods for the detection and analyses of nucleic acid sequences, where the target sequences can be amplified using PCR, proteins are not amplifiable by current techniques. Proximity ligation was developed as a protein detection method that converts protein detection into target DNA detection after PCR amplification. The PCR amplification provides an increase in protein detection sensitivity.

Proximity ligation is a technique where two oligonucleotide-tagged recognition elements (such as antibodies or DNA aptamers) bind to an analyte (i.e. a protein) in solution. Binding to the analyte brings the oligonucleotides into close proximity with one another, and the oligonucleotides can be ligated. After ligation, the resulting DNA sequence is amplified by PCR, and the products are analyzed by real-time PCR (qPCR), which provides an assay readout with wide dynamic range (i.e. quantitative over several orders of magnitude).

Current methods or real-time PCR for proximity ligation may lead to amplification of nonspecific sequences and generation of PCR byproducts, which interfere with the overall sensitivity or efficiency of the assay. Byproducts may react with Taqman probes or DNA-binding dyes used for qPCR, increasing the background signal. Accurate qPCR measurements also require careful assay design and expensive equipment.

### SUMMARY

This disclosure is directed to methods, kits, and devices for enhancing the sensitivity of proximity ligation assays. In embodiments, the methods described herein comprise using size separation techniques to enhance the sensitivity of a proximity ligation assay. A method for enhancing the sensitivity of a proximity ligation assay comprises detecting and/or quantitating one or more analytes in a sample by detecting and/or quantitating an analyte specific amplification product comprising separating the analyte specific amplification product by size from a mixture of amplification products, wherein the mixture of amplification products is formed by amplification of a ligated probe formed when at least two different proximity probes each specifically bind the analyte. In some embodiments, ligation products are amplified by standard PCR methods, and the amplification products are analyzed by a size separation technique, for example, by capillary electrophoresis.

In still other embodiments, the methods herein include design of PCR primers for amplifying ligation products. Primers that amplify a product of a specific length, or create fewer nonspecific amplification products, provide increased sensitivity. In some embodiments, the PCR primers provide for an analyte specific amplification product of at least 100 base pairs. In some embodiments, the number of amplification cycles is adjusted to provide for an increase in the analyte specific amplification product and/or decrease in the nonspecific amplification products. The sensitivity of methods of the invention is also improved by increasing the efficiency of the ligation reaction. In some embodiments, a method further comprises phosphorylating each oligonucleotide probe with a polynucleotide kinase before ligation of the probes. In other embodiments, the proximity probes and/or connector oligonucleotide comprise a chain terminating nucleotide.

Kits that include compositions for increasing the sensitivity of a proximity ligation assay are also provided herein. The kits include one or more reagents useful for PCR amplification, and methods and devices for effective size separation of amplification products.

### BRIEF DESCRIPTION OF THE DRAWINGS

FIG. 1 is a graphical representation of the limits of detection for various protein detection methods.

FIG. 2 is a capillary electropherogram showing an overlay of the signal for the detected protein versus replicate control samples.

FIG. 3A represents a “gel-like” electropherogram illustrating a specific PCR product amplified with two different sets of primers.

FIG. 3B is a graphical representation showing a relative comparison of PCR products generated with two different sets of primers.

FIG. 4A is a graphical representation showing a relative comparison of PCR products generated with unphosphorylated and phosphorylated oligonucleotides.

FIG. 4B is a graphical representation showing a relative comparison of PCR products generated using commercially phosphorylated oligonucleotides (obtained from Operon Biotechnologies) and in vitro phosphorylated oligonucleotides.

FIG. 5 is a graphical representation showing a relative comparison of PCR products generated using newly designed primers and various numbers of PCR cycles for amplification.

### DETAILED DESCRIPTION

Various embodiments of the present methods will be described in detail with reference to the drawings, wherein like reference numerals represent like parts throughout the several views. Reference to various embodiments does not limit the scope of the methods, which is limited only by the scope of the claims attached hereto. Additionally, any examples set forth in this specification are not intended to be limiting and merely set forth some of the many possible embodiments for the claimed methods.

All publications and patent applications in this specification are indicative of the level of ordinary skill and are incorporated herein by reference in their entireties.

As used herein, the term “analyte” refers to a particular biological compound or biomolecule, such as a protein, for example, that is present in a biological sample and is targeted for detection by the methods described herein.

The terms “deoxyribonucleic acid” and “DNA” as used herein mean a polymer composed of deoxyribonucleotides.

The terms “ribonucleic acid” and “RNA” as used herein mean a polymer composed of ribonucleotides.

The term “DNA aptamer” refers to oligonucleotides that have been selected for binding to a target moiety from a population of random sequences, typically through a combinatorial search. The selected oligonucleotides have the ability to recognize a specific target moiety. Target moieties include nucleic acids, proteins, peptides or molecules.

“Ligation” or “DNA ligation” refers to joining DNA fragments together with covalent bonds through the action of an enzyme, such as T4 DNA ligase, for example. More specifically, DNA ligation involves creating a phosphodiester bond between the 3′ hydroxyl of one nucleotide and the 5′ phosphate of another.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The term “oligonucleotide” as used herein means a polymer composed of either DNA or RNA.

The term “polymerase” refers to an enzyme that links individual nucleotides together into a long strand, using another strand as a template. There are two general types of polymerase—DNA polymerases (which synthesize DNA) and RNA polymerase (which makes RNA). Within these two classes, there are numerous sub-types of polymerase, depending on what type of nucleic acid can function as template and what type of nucleic acid is formed. For example, for amplification of nucleic acid sequences by methods such as PCR, Taq polymerase, a thermostable polymerase obtained from the Thermus aquaticus organism, is commonly used. Taq polymerase can amplify a 1 kb strand of DNA in 30 to 60 seconds at a temperature of about 72° C.

“Polymerase chain reaction” or “PCR” refers to a technique used to amplify (or create multiple copies of), well-defined regions of a DNA strand (typically up to about 10 kb in length), such as a gene, or part of a gene, or DNA fragment, for example. PCR reactions, conditions and optimization parameters are well known to those of skill in the art. The products of PCR are referred to as amplification products and, typically, are oligonucleotides of a specific size. The size of the amplification product depends on the size of template and specificity of the primers utilized. The term “thermal cycler” refers a device that heats and cools PCR reaction mixtures to the precise temperature required for each step of the reaction. PCR assays typically employ a specific number of cycles in the thermal cycler for amplification. The term “real time PCR” (quantitative PCR, or qPCR) refers to a method to rapidly measure the quantity of PCR product in real time, and provides an indirect method for quantitatively measuring starting amounts of DNA, cDNA or RNA. This method typically uses fluorescent dyes and/or probes to measure the amount of amplified product in real time during thermal cycling, and can be used in conjunction with conventional PCR methods.

The term “proximity probe” as used herein refers to a moiety that binds to an analyte and is detectable using oligonucleotide amplification methods. In some embodiments, a proximity probe comprises an analyte recognition moiety and an oligonucleotide probe. In some embodiments, an analyte specific amplification product can be formed when at least two proximity probes specific for the analyte are bound to the analyte and the oligonucleotide probe of each proximity probe is ligated to one another to form a ligated probe that is amplified.

In the context of this description, the term “sensitivity” refers to the smallest amount of PCR product (i.e. the limit of detection) detectable by the readout method used for a proximity ligation assay. The term “limit of detection” (or LOD) refers to the minimum concentration of a substance being analyzed in an assay, that has a 99 percent probability of being identified. An increase in the sensitivity of the assay implies a lowering of the limit of detection. The terms “sensitivity” and “efficiency” are used interchangeably herein.

### Methods for Increasing the Sensitivity of Proximity Ligation Assays

The present description provides kits and methods for increasing the sensitivity of proximity ligation assays. In embodiments, the current methods include modifications of proximity ligation assays so as to increase the sensitivity of the assay readout, and/or by reducing the background of the assay readout. The methods described herein include modifications, such as use of size separation for the assay readout, for example. In other embodiments, PCR-based methods are used to amplify proximity ligation products and size separation methods are used to analyze these products. In embodiments, the methods described herein also include PCR primers to enhance the sensitivity of proximity ligation, as well as increase the level of multiplexing. In other embodiments, oligonucleotides are phosphorylated before the reaction, thereby increasing the sensitivity of the assay.

In embodiments, the present description provides methods for improving the sensitivity of proximity ligation assays. Proximity ligation assays are methods to detect an analyte using a proximity probe that specifically bind the analyte and can be detected by oligonucleotide amplification methods.

A sample containing one or more analytes is contacted with a proximity probe that includes an analyte recognition moiety and an oligonucleotide probe. When two proximity probes bind to the same analyte, the oligonucleotide probes of each proximity probe are brought in close contact to one another so that the oligonucleotide probes can be ligated to one another to form the ligated probe. Optionally, in the presence of a connector oligonucleotide that has portions complementary to both of the oligonucleotide probes, the ligated probe is amplified using either real time or quantitative PCR (qPCR).

However, the use of qPCR requires expensive equipment and the assay must be designed very carefully, to avoid interference from nonspecific PCR byproducts. Because qPCR focuses on the total amount of DNA product generated, nonspecific products generate high signal in the assay readout, which contributes to the total background. It therefore becomes difficult to distinguish between the assay readout signal for specific ligation products and the signal for nonspecific products. This has a negative impact on the sensitivity of the reaction. In the methods described herein, the amplified ligation products are analyzed by methods in addition to and/or other than qPCR, and this modification increases the sensitivity of the assay.

In some embodiments, a method for enhancing the sensitivity of a proximity ligation assay comprises detecting and/or quantitating one or more analytes in a sample by detecting and/or quantitating one or more specific amplification products comprising separating at least one analytic specific amplification product from a mixture of amplification products by size; wherein the mixture of amplification products is formed by amplifying a ligated probe formed when at least two different proximity probes each specifically bind the same analyte. In some embodiments, each proximity probe specific for the analyte comprises an analyte recognition moiety, and an oligonucleotide probe and wherein the at least two different proximity probes specific for the same analyte differ from one another by recognizing a different portion of the analyte and each oligonucleotide probe of the at least two different proximity probes can be ligated to one another to form the ligated probe.

In some embodiments of methods described herein, PCR primers are designed to provide an amplification product of at least 50 base pairs to about 10,000 base pairs. In some embodiments, the PCR primers are designed to provide an amplified product large enough to be readily separated by size from non-specific amplification products. In an aspect, the target amplified product is at least 10 bp longer than the non-specific amplification product, such that the target amplified product can be readily distinguished from the non-specific amplification product by standard techniques.

In another embodiment of the methods described herein, the oligonucleotide probe of each proximity probe is phosphorylated such that the specific activity of the phosphorylation is increased above the activity observed for commercially phosphorylated probes. In some embodiments, the oligonucleotide probe is phosphorylated using a polynucleotide kinase before ligation of the probe.

In some embodiments, the ligated probe is formed by hybridizing a connector oligonucleotide to each of the oligonucleotide probes of the at least two different proximity probes, wherein the connector oligonucleotide comprises a sequence complementary to each of the oligonucleotide probes, and then ligating each of the oligonucleotide probes to one another using a ligase.

In some embodiments of the methods described herein, the oligonucleotide probes and connector oligonucleotide comprise chain terminating nucleotides.

In further embodiments of the methods as described herein, the ligated probe is amplified using polymerase chain reaction and at least 26 cycles of amplification. In some embodiments, the amplification cycles are about 28 to 1000 cycles. In further embodiments, the number of cycles selected is that in which the specific amplification products are increased at least about 3 fold as compared to specific amplification products generated with 25 cycles or less.

### Analytes

The kits and methods of the disclosure as described herein provide for increased sensitivity in detecting and/or quantitating one or more analytes in a sample. Analytes can include any molecule that can be recognized and/or bound by an analyte recognition moiety. Analytes include proteins, peptides, DNA, RNA, microbes, viruses, receptors, ligands, metal ions, polymers, minerals and other organic or inorganic compounds. In some embodiments, the analyte is present at a concentration of about 0.5 pg/ml to about 50 ng/ml. The methods described here allow detection of the protein PDGF at less than 1 pg/ml, and could be extended either to other proteins or lower ranges of detection. In some embodiments, methods as described herein provide improved detection of, for example, HIV or other viruses, or cancer biomarkers.

The samples can include biological samples such as blood, serum, plasma, urine, tissue, sweat, saliva, microbial or viral cultures and the like. Samples can also include soil samples, water samples, and the like.

### Proximity Probes

As described herein, the methods of the disclosure provide for detection and/or quantitation of one or more analytes using at least two different proximity probes. A proximity probe comprises an analyte recognition moiety and an oligonucleotide probe. In some embodiments, at least two different proximity probes are utilized, wherein each of the different proximity probes binds to different portions of the analyte. Each of the different proximity probes has an analyte recognition moiety and an oligonucleotide probe that differ from one another. One proximity probe should provide an oligonucleotide with a free 5′ end and the other proximity probe should provide an oligonucleotide with a free 3′ end, such that the two oligonucleotide probes can be ligated together.

Analyte recognition moieties include antibodies, aptamers, peptides, avimers, affibodies, ligands (such as peptides, proteins, or small molecules) that bind to a receptor as the analyte, oligonucleotides that hybridize to a specific DNA or RNA analyte, or receptors that bind to a ligand or small molecule as an analyte. In some embodiments, the analyte recognition moiety comprises an antibody or antigen binding fragment thereof, and/or an aptamer. At least two different proximity probes bind to different portions of the analyte, and may be the same or different type of recognition moiety. For example, one analyte recognition moiety can be an aptamer specific for an analyte, and the other analyte recognition moiety can be an antibody specific for the same analyte. In another example, two identical recognition moieties could be used to detect an analyte which is a dimer or multimer (that is, an analyte containing at least two repeated units).

In some embodiments, the oligonucleotide probes on the two different proximity probes also have an oligonucleotide sequence and/or orientation that differs from one another. In some embodiments, the first proximity probe comprises an oligonucleotide probe coupled to the analyte recognition moiety through its 5′ end leaving a free 3′ end and the second proximity probe comprises an oligonucleotide probe coupled to the analyte recognition moiety through its 3′ end leaving a free 5′ end. In some embodiments, a proximity probe can comprise an oligonucleotide probe that is phosphorylated at its free end.

In some embodiments of the methods, the oligonucleotide probe portion of the proximity probe is phosphorylated before the probes are ligated. The probes are phosphorylated such that the specific activity of the probes is increase over the activity observed for commercially phosphorylated oligo probes. In some embodiments, the oligonucleotide probe is phosphorylated with a kinase, such as polynucleotide kinase. It is expected that this will improve assay sensitivity as the assay signal is increased. It is believed that the ligation reaction is improved because the ligase requires a 5′ phosphate group. In some embodiments, methods of the disclosure comprise phosphorylating the oligonucleotide probes of the proximity probe with a kinase prior to ligation.

### Forming the Ligated Product

In the methods of the invention an analyte specific amplification product is detected and/or quantitated. An analyte specific amplification product is separated and identified from a mixture of amplification of products. The amplification product or mixture of products is formed by amplifying a ligated product formed when two proximity probes are bound to the same analyte and the oligonucleotide probe portions of each proximity probes are brought into close proximity to one another and are ligated together.

As discussed previously, in some embodiments, one of the proximity probes comprises an oligonucleotide probe that has a free 3′ end and the other proximity probe bound to the same analyte comprises an oligonucleotide probe with a free 5′ end. The oligonucleotide probes of each of the bound proximity probes can be ligated together to form the ligated probe. In some embodiments, sensitivity of the assay is enhanced if the oligonucleotide probe is phosphorylated at the 5′ position.

In some embodiments, a connector oligonucleotide may be utilized to couple each of the oligonucleotide probes of the bound proximity probes together. In some embodiments, the connector oligonucleotide hybridizes to a portion of one of the oligonucleotide probes and to a portion of the other oligonucleotide probe. In some embodiments, the connector oligonucleotide has at least three non-complementary nucleotides at the 3′ and/or 5′ end, so the ends do not hybridize to either of the oligonucleotide probes. In some embodiments, the oligonucleotide probes and the connector oligonucleotide comprise chain termination nucleotides, such as dideoxy nucleotides.

### PCR Amplification and Primers

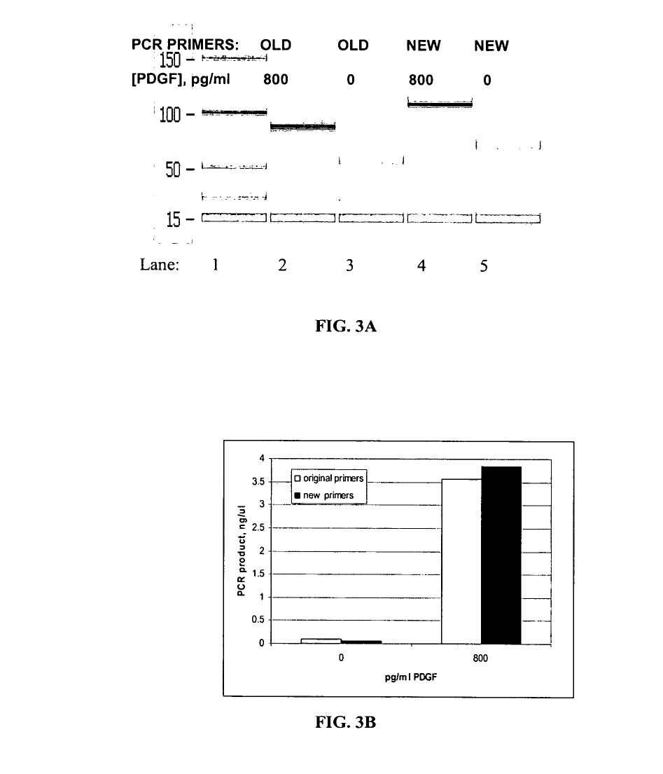

The methods described herein enhance the sensitivity and multiplex capability of proximity ligation assays, by using PCR primers to amplify the ligated product. In embodiments, the primers optimize the assay, and primers that result in longer PCR fragments provide better size discrimination over nonspecific amplification products. When amplified ligation products are analyzed by capillary electrophoresis, both the original PCR primers and the designed PCR primers give rise to low-molecular weight bands in the electropherogram corresponding to nonspecific products (often a class of these nonspecific products is referred to as “primer dimer”; see FIG. 3A, for example). In a qPCR analysis, these nonspecific products would contribute to the overall background signal and interfere with the signal from the specific product. However, with capillary electrophoresis, the band produced by the nonspecific product is only relevant (i.e. contributes to the background signal) if the band is in the region of the size expected for the actual product. Because the larger PCR products produced from the designed PCR primers are more easily distinguishable (by their larger size) from shorter, nonspecific products, using newly designed primers helps reduce background from nonspecific products and thereby enhances the sensitivity of the proximity ligation assay.

In embodiments, changing the length of the amplified products (by using PCR primers of different sequences) allows a much higher degree of multiplexing, relative to qPCR methods. This modification makes the assay adaptable to multiplexed detection. For example, different sizes of PCR products as a result of primers design may provide a method of detection of multiple proteins in a mixture. For example, different PCR products of 100, 120, 140, 160, 180, and 200 bp could correspond to the concentrations of six different proteins measured in parallel. Amplification products could range in size from around 50 bp to several kilobases, and any size could be used which allows both efficient amplification and efficient size separation. PCR primers with non-complementary 5′ ends could be used to create longer amplification products from a shorter ligated probe (e.g., if a ligated probe is only 60 nucleotides long, extending the 5′ end of each PCR primer by 10 nucleotides would allow creation of a 80 bp amplification product.) Changing the primer sequence could easily allow a person of skill to customize the expected sizes of PCR products for a given sequence or set of sequences to be amplified.

Methods of designing primers are known to those of skill in the art. In some embodiments, the primers hybridize to the ligated oligonucleotide probe so that the amplification of the ligated probe provides an analyte specific amplification product of at least 100 base pairs. In some embodiments, the PCR primers comprise a sequence for forward primer 5′GGCTGAGTATGTGGTCTATGTCG3′ (SEQ ID NO: 1) and for a reverse primer 5′CTTGCAGTGCCCTGAGTAAGA3′ (SEQ ID NO: 2).

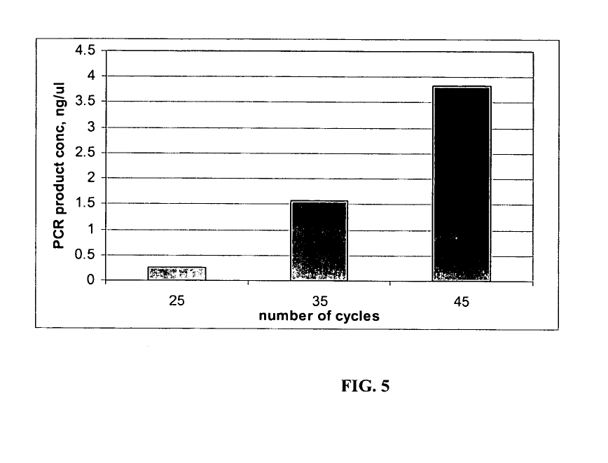

In some embodiments, increased sensitivity of the assay can also be obtained by utilizing at least 30 PCR cycles, or about 28 to 45 cycles, as shown in FIG. 5. Higher numbers of cycles may increase both the specific and nonspecific amplification products, but these can be separated by a size separation technique, such as standard method known to those of skill in the art.

In other embodiments, the temperature of the primer annealing/extension step of the PCR reaction is varied from about 50 to 72° C. Lowering the annealing temperature of the PCR reaction can result in an increase in nonspecific priming, which usually leads to amplification products of the incorrect size. These nonspecific amplification products would be expected to raise the background signal in a qPCR readout of the assay, but will not increase the background signal of the size-separation readout of the assay, which can accurately detect the specific amplification product in a mixture of specific and nonspecific amplification products. Lowering the annealing temperature may also increase the amount of the specific amplification product. Thus, in an embodiment where the amplification products are separated by size, lowering the annealing temperature of the PCR reaction can result in an increase in signal without raising the background signal. In some embodiments, the PCR amplification of the ligated product is conducted at a temperature of 58 to 62° C.

### Assay Readout

The use of size separation improves the sensitivity of the proximity ligation assay by at least two-fold (i.e. the limit of detection of the assay is reduced by a factor of two). In typical proximity ligation assays, sensitivity is limited by background, such as impurities from probe synthesis, connector synthesis, or primer synthesis, or incorrect products from nonspecifically bound PCR primers, for example. These impurities interfere with the subsequent qPCR analysis, and make it difficult to distinguish between specific ligation products and nonspecific products. Size separation methods, such as capillary electrophoresis, for example, can distinguish between DNA fragments that differ in size by one, or only a few base pairs. Nonspecifically amplified products of varying sizes are easily distinguished from the specific products of the correct, expected size. Using size separation methods, smaller quantities of a protein can be detected, relative to the amount detected using standard proximity ligation assays.

The present description provides methods for enhancing the sensitivity of a proximity ligation assay by modifying the assay readout method. In embodiments, the modification involves using a size separation method for the readout, such as capillary electrophoresis, for example. Ligation products are generated as described above. In some embodiments, instead of amplifying the ligation products using real-time or qPCR methods, the products are amplified using a standard thermal cycler, as typically used in PCR methods. After a set number of cycles are complete, the products are analyzed directly, using a size separation technique such as capillary electrophoresis, for example. Other electrophoretic or size separation methods can also be used in conjunction with this method. In alternate embodiments, the size separation analysis is used to analyze ligation products amplified by qPCR methods.

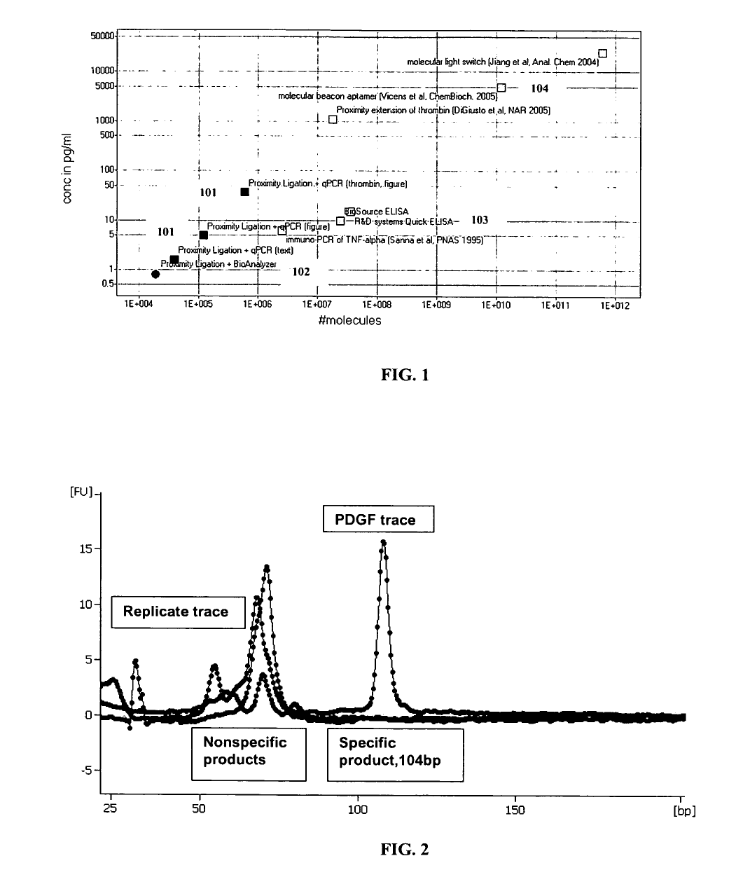

The size selection modification lowers the background signal of the assay and provides flexibility to vary conditions and increase the signal-to-background ratio, thereby lowering the limit of detection. FIG. 1 demonstrates the limits of detection for various methods for protein detection. The elements 101 (filled squares) in FIG. 1 represent results from proximity ligation assays performed using qPCR as reported in Fredriksson et al., Nature Biotech. 20: 473-477 (2002), whereas 102 (filled circle) represents proximity ligation assays performed using the methods described herein. Light gray squares represent protein detection by other methods such as commercial ELISA, in 103, and other methods using DNA aptamers, in 104. FIG. 1 demonstrates that proximity ligation is a more sensitive method for protein detection, relative to existing methods such as ELISA. Furthermore, element 102 in FIG. 1 clearly indicates that proximity ligation combined with size separation (using the Agilent BioAnalyzer, Agilent Technologies, Inc., Palo Alto, Calif.), as described herein, shows increased sensitivity over proximity ligation using qPCR.

### Methods for Further Enhancement of Assay Sensitivity

In embodiments, the sensitivity of the proximity ligation assays can be further enhanced by combining the size selection modification with variation of other experimental conditions. Because a size separation method such as capillary electrophoresis reduces the background signal (to low or almost nonexistent), the sensitivity assay can be increased to a greater extent than is feasible with qPCR. For example, a given modification may increase the levels of both the correct PCR products (signal) and incorrect PCR products (background) equally in a qPCR assay, and thus the modification would appear to have no benefit in qPCR. However, size separation further distinguishes the correct PCR products from the incorrect products, and in this case the increase in signal may be recognized as a significant increase in sensitivity. In one aspect, the sensitivity of the assay is increased by changing the concentration of the enzymes used in the assay. For example, increasing the amount of ligase of Taq polymerase (such as doubling the concentration, or increasing the concentration ten-fold) may increase the amount of amplification product. In another aspect, the sensitivity of the assay is enhanced by changing the ligation time. Increasing the ligation time from 5 minutes as described in Fredriksson et al., Nature Biotech. 20: 473-477 (2002) to 20 minutes, or even longer, increases the amount of ligated product and thus can increase the sensitivity. In yet another aspect, the assay is made more sensitive or efficient by modifying the number of thermal/PCR cycles (see for example, FIG. 5), and/or the temperature at which PCR is performed as described herein. Such optimizations are not limited to the proximity ligation methods described herein, and can be easily extended to other proximity ligation methods.

In some embodiments, the methods as described herein provide for detection of one or more analytes at about 0.05 pg/ml to about 50 ng/ml. In other embodiments, the methods of the invention with increased sensitivity can detect about 10,000 molecules of analyte up to about 100,000,000 molecules of analyte.

### Kits for Increasing Sensitivity of Proximity Ligation Assays

In embodiments, the present disclosure includes kits for increasing the sensitivity of proximity ligation assays to detect an analyte. In embodiments, the kits described herein contain stock solutions of reagents necessary for PCR amplification of ligation products, and reagents necessary for effective size separation of amplified ligation products (for example, reagents used for electrophoresis of the PCR products). The stock solutions of the kit can be used to make various different concentrations of the necessary reagents. The kit may also contain instructions providing information on the use of PCR amplification and size separation, in conjunction with a proximity ligation assay. In embodiment, the kits may further contain reagents needed for in vitro phosphorylation of oligonucleotides prior to the ligation reaction, and subsequent PCR-based amplification. The kit may also include positive and negative control reagents, such as standard aliquots of analyte, ligation control DNAs, PCR amplification controls, etc., which could be used to calibrate the assay or evaluate the success of the different steps of the protocol.

### EXAMPLE 1

### Assay Readout by Capillary Electrophoresis

For purposes of this example, the proximity ligation assay is used to detect the presence of a dimer of a platelet-derived growth factor B-chain protein (PDGF-BB). The examples that follow are provided by way of illustration only. The methods described herein are applicable to detection of other analytes, e.g. proteins, in other biological systems.

Proximity probes were generated from DNA aptamers that specifically bind the PDGF dimer, by adding oligonucleotide probe sequence (approximately 40 nt in length) to the 5′ or 3′ ends of the aptamers, as described in Fredriksson et al., Nature Biotech. 20: 473-477 (2002). The sequence for the proximity probe pair is as follows:

(SEQ ID NO: 1)

5′-
TACTCATGGGCACTGCAAGCAATTGTGGTCCCAATGGGCTGAGTA-3′

(SEQ ID NO: 2)

3′-
TATGAGTCGGGTAACCCTGGTGTTAACGAACGTCACGGGACTCAT-5′

The proximity probe pairs are subjected to sequence extension, prior to ligation and hybridization with a connector oligonucleotide. These sequence extensions have the following sequences, with SEQ ID NO: 3 corresponding to the sequence extension for a probe with the sequence in SEQ ID NO: 1, and SEQ ID NO: 4 corresponding to the sequence extension for a probe with the sequence in SEQ ID NO: 2:

(SEQ ID NO: 3)

5′-TGTGGTCTATGTCGTCGTTCGCTAGTAGTTCCTGGGCTGCAC-3′

(SEQ ID NO: 4)

3′-TCTTGTCGCGCGTAGCCCCCTTAAGATGCGGAGCT-5′

A sample containing 0.8 pg/mL of PDGF was incubated with a pair of proximity probes, in a volume of 5 μL at 37° C. for about 15 min. to about 1 hour. The ends of the proximity probe were then ligated by enzymatic ligation, using a connector oligonucleotide, approximately 20 nt in length, as the template for ligation, as described in Fredriksson et al., Nature Biotech. 20: 473-477 (2002). The connector oligonucleotide was designed to hybridize to the free 5′ and 3′ ends of each of the proximity probes bound to the protein. The sequence for the connector oligonucleotide is shown below:

| | |
|---|---|
| 3′-TTTACCCGACGTGAGCTCCGCATAAA-5′ | (SEQ ID NO: 5) |

Ligation products were then amplified using a regular thermal cycler and standard PCR methods. After 30 to 45 cycles, the amplified products were analyzed directly using an Agilent Bioanalyzer capillary electrophoresis system. A capillary electropherogram (FIG. 2) is generated as the readout.

FIG. 2 shows an overlay of the signal traces from PDGF versus the signal traces from replicate samples containing no PDGF. All the sample traces show nonspecific amplification products at 50-70 bp. However, the trace for the sample containing PDGF (i.e. the analyte) was the only one showing a peak at 104 bp, with background signal in that region being zero. This corresponds to less than 20,000 molecules of PDGF, which is a two-fold improvement in the limit of detection, when compared to the same assay performed using qPCR.

### EXAMPLE 2

### Designing New Primers

In order to increase sensitivity of the assay, the 104 bp sequence corresponding to PDGF (as identified in Example 1) is amplified by PCR, using PCR primers comprising the following sequences:

| | |
|---|---|
| New Forward primer: | |
| 5′ GGCTGAGTATGTGGTCTATGTCG 3′ | (SEQ ID NO: 6) |
| New Reverse primer: | |
| 5′ CTTGCAGTGCCCTGAGTAAGA 3′ | (SEQ ID NO: 7) |

### The newly designed primers are compared to primers as described in Fredriksson et al., Nature Biotech. 20:473-477 (2002):

| | |
|---|---|
| Forward primer: | |
| 5′ ATGTGGTCTATGTCGTCGTTCG 3′ | (SEQ ID NO: 8) |
| Reverse primer: | |
| 5′ TGAGTAAGAACAGCGCGCAT 3′ | (SEQ ID NO: 9) |

Proximity ligation products from parallel experiments were amplified either using the above designed primers (NEW), or the original primers used in the experiment as described in Fredriksson et al., Nature Biotech. 20: 473-477 (2002) (OLD). Replicate samples containing no protein were also similarly amplified. The amplified ligation products were then analyzed by capillary electrophoresis using the Agilent BioAnalyzer system. A “gel-like” electropherogram (FIG. 3) was generated as the assay readout.

FIG. 3A shows a gel-like electropherogram illustrating the PCR product from PDGF and replicate samples, amplified with two different primer sets. Both primer sets produce similar amounts of product at 56 bp (lane 3) and 70 bp (lane 5), which are byproducts of nonspecific amplification. In a qPCR method, these bands would contribute to the overall background signal and interfere with the signal from the actual analyte. However, because size separation methods such as capillary electrophoresis allow detection of an amplified product of a specific size, the analyte specific amplification product is easy to identify, as the 104 bp band (lane 4). Using designed PCR primers, the band at the expected molecular weight is amplified over smaller fragments only when the analyte is present (in contrast to the band at 84 bp produced by amplification with the old primers in the absence of analyte).

A graphical quantitation from the electropherogram is shown in FIG. 3B. As shown in FIG. 3B, the designed primers produce a larger amount of the specific PCR product in the presence of the analyte (black bar on the right) and a smaller amount of the correct product in the absence of the analyte (black bar on the left); thus the signal to noise ratio is significantly increased.

### EXAMPLE 3

### Phosphorylation of Oligonucleotides

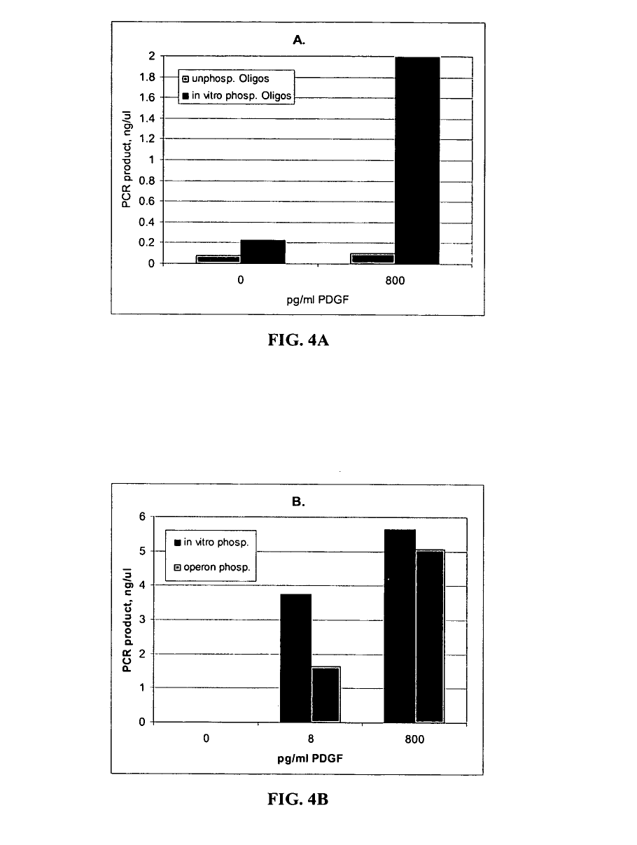

To determine the effect of phosphorylation of the oligonucleotide probe portion of the proximity probe on the efficiency of the methods as described herein, the probe oligonucleotides were phosphorylated. Because DNA ligase requires the presence of a 5′-phosphate group on the oligonucleotides, phosphorylation of the oligonucleotides can increase the efficiency of ligation (FIG. 4A). Further, when the oligonucleotides are phosphorylated in vitro, using polynucleotide kinase, the assay signal is considerably increased over the assay signal obtained using commercially phosphorylated oligonucleotides (FIG. 4B). While not meant to limit the invention, it is believed that incomplete phosphorylation of the commercially phosphorylated oligonucleotides contributes to inefficiency of the ligation reaction. The addition of a polynucleotide kinase increases production of the ligated product and can increase the sensitivity of assay, including the qPCR version.

FIG. 4A shows the increase in signal (amount of specific PCR product) gained using oligonucleotides phosphorylated in vitro (black bars) vs. unphosphorylated oligonucleotides (grey bars). Note that, because of the presence of Taq polymerase in the reaction, which can add nucleotides to contribute the 5′ phosphate to the oligonucleotide, unphosphorylated oligonucleotides do show a small signal.

In FIG. 4B, results from parallel assays using oligonucleotides phosphorylated in vitro (black bars) vs. commercially synthesized phosphorylated oligonucleotides (Operon Biotechnologies, Inc., Huntsville, Ala.). The signal for in vitro phosphorylated oligonucleotides is considerably greater, when compared to the assay using the Operon phosphorylated oligonucleotides.

### EXAMPLE 4

To determine the effect of varying the number of thermal cycles on the efficiency of the methods described herein, proximity ligation products were amplified with the designed primers in parallel experiments. For each experiment, a different number of cycles were used. FIG. 5 shows a graphical representation of this experiment, comparing the amount of PCR product formed with the number of thermal cycles used for amplification. It can be seen from FIG. 5 that a 6-fold increase in amplification is seen when the number of cycles is increased from 25 to 35. An even greater increase (of about 15-fold) is seen when the number of cycles is increased from 25 to 45. Therefore, the sensitivity and efficiency of the methods described herein are improved by increasing the number of thermal cycles.

The various embodiments and examples described above are provided by way of illustration only and should not be construed to limit the present methods. Those skilled in the art will readily recognize various modifications and changes that may be made to the described methods without following the example embodiments and applications illustrated and described herein, and without departing from the true spirit and scope of the present methods, which is set forth in the claims attached hereto.

1. A method for enhancing the sensitivity of a proximity ligation assay, comprising detecting and/or quantitating one or more proteins in a sample by detecting and/or quantitating one or more protein specific amplification products comprising separating at least one protein specific amplification product from a mixture of amplification products by size, wherein the mixture of amplification products is formed by amplification of a ligated probe formed when at least two different proximity probes each specifically bind the same analyte and a connector oligonucleotide binds to each of the at least two different proximity probes.

2. The method of claim 1, wherein amplification of the ligated probe comprises amplifying the ligated probe with at least two primers and at least 28 amplification cycles to form the mixture of amplification products.

3. The method of claim 1, wherein each proximity probe comprises an a protein recognition moiety and an oligonucleotide probe, and wherein the at least two proximity probes specific for the same protein differ from one another by recognizing a different portion of the protein and each oligonucleotide probe of each of the at least two different proximity probes can be ligated to one another to form the ligated probe.

4. The method of claim 3, further comprising phosphorylating each oligonucleotide probe with a polynucleotide kinase before ligation of the probes.

5. The method of claim 2, wherein amplification of the ligated probe comprises PCR.

6. The method of claim 5, further comprising phosphorylating each oligonucleotide probe with a polynucleotide kinase before ligation of the probes.

7. The method of claim 1, wherein separation of the protein specific amplification product from the mixture of amplification products comprises electrophoresis.

8. The method of claim 7, wherein electrophoresis is capillary electrophoresis.

9. The method of claim 1, wherein the limit of detection for detecting and/or quantitating one or more proteins is improved by at least 2-fold as compared to a method using quantitative PCR.

10. The method of claim 1, wherein the background of detecting and/or quantitating one or more proteins is decreased by at least 2-fold as compared to a method using quantitative PCR.

11. The method of claim 2, wherein the primers provide for an amplification product of at least 100 base pairs.

12. The method of claim 11, wherein the primers provide for an amplification product of about 100 bp to about 10000 bp.

13. The method of claim 3, wherein the ligated probe is formed by hybridizing the connector oligonucleotide to each of the oligonucleotide probes of the at least two different proximity probes, wherein the connector oligonucleotide comprises a sequence complementary to each of the oligonucleotide probes and then ligating each of the oligonucleotide probes to one another using a ligase.

14. The method of claim 13, wherein the connector oligonucleotide and each oligonucleotide probe comprise chain terminating nucleotides.

15. The method of claim 14, wherein the chain-terminating nucleotides are dideoxynucleotides.

16-22. (canceled)

23. A method for enhancing the sensitivity of a proximity ligation assay, comprising detecting and/or quantitating one or more analytes in a sample by detecting and/or quantitating one or more analyte specific amplification products comprising separating at least one analyte specific amplification product from a mixture of amplification products by size, wherein the mixture of amplification products is formed by amplification of a ligated probe formed when at least two different proximity probes each specifically bind the same analyte and a connector oligonucleotide binds to each of the at least two different proximity probes, wherein the one or more analytes can be detected and/or quantitated in the samples at a concentration of 0.05 pg/ml to 50 ng/ml.


SEQUENCE LISTING (9 sequences)

SEQ ID NO: 1 - DNA, 45 bp, Artificial
  primers
tactcatgggcactgcaagcaattgtggtcccaatgggctgagta

SEQ ID NO: 2 - DNA, 45 bp, Artificial
  primers
tatgagtcgggtaaccctggtgttaacgaacgtcacgggactcat

SEQ ID NO: 3 - DNA, 42 bp, Artificial
  primers
tgtggtctatgtcgtcgttcgctagtagttcctgggctgcac

SEQ ID NO: 4 - DNA, 35 bp, Artificial
  primers
tcttgtcgcgcgtagcccccttaagatgcggagct

SEQ ID NO: 5 - DNA, 26 bp, Artificial
  primers
tttacccgacgtgagctccgcataaa

SEQ ID NO: 6 - DNA, 23 bp, Artificial
  primers
ggctgagtatgtggtctatgtcg

SEQ ID NO: 7 - DNA, 21 bp, Artificial
  primers
cttgcagtgccctgagtaaga

SEQ ID NO: 8 - DNA, 22 bp, Artificial
  primers
atgtggtctatgtcgtcgttcg

SEQ ID NO: 9 - DNA, 20 bp, Artificial
  primers
tgagtaagaacagcgcgcat